(19) United States
(12) Patent Application Publication
(10) Pub. No.: US 2009/0131653 A1
(43) Pub. Date: May 21, 2009

(54) Generation of Phosphorus Oxychloride as by-Product from Phosphorus Pentachloride and DMF and its Use for Chlorination Reaction by Converting Into Vilsmeier-Haack Reagent

(75) Inventors: Rakesh Ratnam, Maharashtra (IN); Aurora Sundeep, Maharashtra (IN); Mohammed Mofizuddin, Maharashtra (IN)

Correspondence Address: THE NATH LAW GROUP, 112 South West Street, Alexandria, VA, 22314, US

(73) Assignee: PHARMED MEDICARE PRIVATE LIMITED, Mumbai (IN)

(21) Appl. No.: 11/919,826

(22) PCT Filed: Apr. 28, 2006
(86) PCT No.: PCT/IN2006/000151

§ 371 Date: Dec. 31, 2007

(30) Foreign Application Priority Data
May 4, 2005 (IN) 545/MUM/2005

(51) Int. Cl. C07H 1/00; C07C 251/30

(52) U.S. Cl. 536/124; 564/278

(57) ABSTRACT

A process is described wherein after formation of first crop of Vilsmeier-Haack reagent by reacting Phosphorus Pentachloride with N,N-dimethylformamide to form a first crop of Vilsmeier reagent as insoluble crystals, a by-product of this reaction, the Phosphorus Oxy-Chloride, reacts with N,N-dimethylformamide to give a second crop of Vilsmeier reagent. This second crop of Vilsmeier reagent is soluble in DMF. This process makes it possible to double the yield of chlorinated substrate, such as sucrose-6-acetate or sucrose-6-benzoate, from the same quantity of Phosphorus Pentachloride.

### TECHNICAL FIELD

The present invention relates to a process and a novel strategy for synthesis of Vilsmeier-Haack reagent and chlorination of sucrose or their derivatives for production of chlorinated compounds including sucrose, 1′-6′-Dichloro-1′-6′-DIDEOXY-β-Fructofuranasyl-4-chloro-4-deoxy-galactopyranoside using said Vilsmeier-Haack reagent.

### BACKGROUND OF THE INVENTION

Strategies of prior art methods of production of 4,1′, 6′ trichlorogalactosucrose predominantly involve use of Vilsmeier-Haack reagent (Vilsmeier reagent) to chlorinate Sucrose-6-ester, mainly Sucrose-6-acetate to form 6 acetyl 4,1′, 6′trichlorogalactosucrose (TGS-6-acetate) or corresponding chlorinated derivative, which is deacetylated in the reaction mixture itself to form 4,1′, 6′ trichlorogalactosucrose (TGS).

When Vilsmeier-Haack reagent is produced from PCl₅, as described by Mufti et al (1983) in U.S. Pat. No. 4,380,476, upon reaction of PCl₅ with the appropriate tertiary amide, the Vilsmeier reagent is produced in the form of crystals insoluble in the reaction mixture which is isolated in solid form by filtration, washed twice with DMF, and twice with diethyl ether and used as chlorinating agent.

It was found, surprisingly, however, that if this POCl₃ generated as a byproduct in the course of reaction is not removed from the reaction mixture, POCl₃ further reacts with the tertiary amide, such as N,N-dimethylformamide, available in the reaction mixture, generating a second POCl₃ type Vilsmeier-Haack reagent which is soluble, and does not precipitate out as other types of Vilsmeier-Haack reagents.

This finding opened up a way for developing improved chlorination method involving Vilsmeier reagent formed from using PCl₅, which is the subject matter of this specification.

### PRIOR ART

Jenner et al (1982) U.S. Pat. No. 4,362,869, have used thionyl chloride for preparation of Vilsmeier reagent

Mufti et al (1983) claimed and described use of Vilsmeier reagent for chlorinating sucrose monoesters. They used Vilsmeier reagent to about 7 to 15 molar equivalents per mole of sucrose monoester. An amount of about 33 moles per mole of monoester was considered as optimal. It was pointed out that it is important that water is prevented from contacting the reagent, which was achieved by drying the monoester solution and fitting the reaction vessel with a drying tube. Vilsmeier reagent was prepared by Mufti et al by reacting DMF with PCl₅ accompanied by vigorous stirring while the temperature was maintained below 50.degree. C. The reaction mixture was stirred at 0.degree. for 1 h and the resulting crystals were filtered off, washed with DMF (2.times), then with diethyl ether and dried under vacuum overnight.

Chlorination reaction involved addition of DMF to the crystals of Vilsmeier reagent and adding to them sucrose mono-acetate solution slowly, maintaining temperature below 20° C., and then heating the reaction mixture for a period of time to 60° C. accompanied by removal of HCl gas by bubbling nitrogen through the reaction mixture and then at 120 degrees for a period of time.

The Vilsmeier chlorination is preferably worked up by neutralisation and hydrolysis with an alcohol/base mixture, e.g. methanolic ammonium hydroxide (2:1 by weight).

The general formula of Vilsmeier reagent, irrespective of source of chlorinating reagent used, remained same as described by Mufti et al i.e. an N,N-dialkyl-(chloromethaniminium) chloride of the general formula:

[XClC═N.sup.+R.sub.2]Cl.sup.−

where R represents an alkyl group, typically a methyl or ethyl group, and X represents a hydrogen atom or a methyl group.

Mufti et al further pointed out that, reagents of this type are prepared by reaction of an inorganic acid chloride with an N,N-dialkylformamide or N,N-dialkylacetamide. The inorganic acid chloride may typically be phosphorous pentachloride, phosgene, or thionyl chloride.

Importance of Vilsmeier reagent lies in the fact that surprisingly this reagent will safely chlorinate in the 4′,1′- and 6′-positions of a sucrose molecule although this class of acidic reagent is known for its specificity as a chlorinator of more active primary hydroxy compounds.

Rathbone et al (1986) in U.S. Pat. No. 4,617,269, Walkup et al (1990) in U.S. Pat. No. 4,980,463, also described use of Vilsmeier reagent formed from Phosphorus pentachloride in the same way as described by Mufti et al.

Thus all the prior art references limit the use of PCl₅ to generate and use the Vilsmeier reagent as DMF insoluble solid crystal form.

### SUMMARY OF INVENTION

Present invention embodies formation of two crops of Vilsmeier-Haack reagent from PCl₅. First crop is obtained when PCl₅ is dissolved in dimethylformamide (DMF) and crystals of Vilsmeier reagent formed precipitate out as a first crop of the reagent. One by-product of this reaction is POCl₃, which, if not removed from the reaction mixture, starts reacting with the excess DMF to form a second crop of Vilsmeier reagent accompanied by and indicated by development of a orange to red color. This second crop of Vilsmeier reagent, however, does not precipitate out as crystals, it remains in dissolved condition and is as much effective in chlorination reactions as any other Vilsmeier reagent developed from PCl₅ or other chlorinating reagents.

In a further embodiment of this invention, it is possible to separate the two crops of Vilsmeier reagent obtainable from PCl₅. It has also been found that it is also possible to use the second crop of Vilsmeier reagent developed from POCl₃ independent from the first crop and use it alone or in combination with Vilsmeier reagent developed from a chlorinating reagent other than PCl₅.

In another embodiment of this invention when both the crops of Vilsmeier reagent were allowed to be formed successively in the same reaction mixture, yield of chlorinated substrate available from same quantity of PCl₅ doubled than the prior art methods wherein the solid crystals of the first crop are separated and used for chlorination. The projected mechanisms of the reactions involved is elucidated in FIG. 1.

In yet another embodiment of this invention, the combined Vilsmeier reagent or Vilsmeier reagent formed from the second crop can be combined with Vilsmeier reagent formed from any other acid chloride and such combinations are also equally effective in performing the chlorination reaction.

### BRIEF DESCRIPTION OF DRAWINGS

FIG. 1: Describes projections on mechanism of reactions involved in formation of twin Vilsmeier reagent from PCl₅.

### DETAILED DESCRIPTION OF THE INVENTION

The Vilsmeier-Haack reaction is widely used for formylations. It can be applied to introduce an aldehyde group on activated aromatic compounds, but many other conversions can be achieved with this technology. In general N,N-dimethylformamide (DMF) and a chlorinating agent such as POCl₃ are used to generate the Vilsmeier-Haack reagent. This reagent gets decomposed when bought in contact with water.

In the context of chlorination of sucrose, particularly in the context of preparation of TGS, use of Vilsmeier reagent has been described in several patents and patent applications.

In this entire specification, including claims, it is understood that a singular also includes plural, unless context indicates otherwise. Thus, for example “an acid chloride” includes one or more of all the known acid chlorides. Further, the examples given are only for the purpose of illustration of the working of this invention and actual chemicals used, their proportions and reaction conditions used are not mentioned to limit the scope of invention. Anything that is equivalent or an adaptation of the claims and obvious to an ordinary person skilled in this art is included within the scope of this specification.

In all prior art methods, Vilsmeier reagent is prepared from PCl₅ by reacting the same with DMF when the reagent separates as crystals which are recovered from the reaction mixture by filtration, dried and used for chlorination reaction.

Quite unexpectedly, it was found that, when the first crop of crystals of Vilsmeier reagent were not removed, after a period of time, the reagent developed orange to reddish color, which was found to be due to formation of a second crop of Vilsmeier reagent by reaction of the by-product POCl₃ with the excess DMF. The said second crop of Vilsmeier reagent, however, does not precipitate out as crystals, it remains in dissolved condition and is as much effective in chlorination reactions as any other Vilsmeier reagent developed from PCl₅ or other chlorinating reagents. Thus, in the method of this invention, the first crop of the Vilsmeier reagent crystals is not separated from the reaction mixture, the second Vilsmeier reagent is allowed to be formed in the same reaction mixture and the combined Vilsmeier reagent can be put to chlorination reaction application. Yield of chlorinated substrate achieved in such a combined Vilsmeier reagent is double than that achieved in prior art method.

If desired, it is possible to separate the two crops of Vilsmeier reagent obtainable from PCl₅, the second crop of Vilsmeier reagent developed from POCl₃ be used independent from the first crop either as alone or in combination with Vilsmeier reagent developed from an acid chloride other than PCl₅.

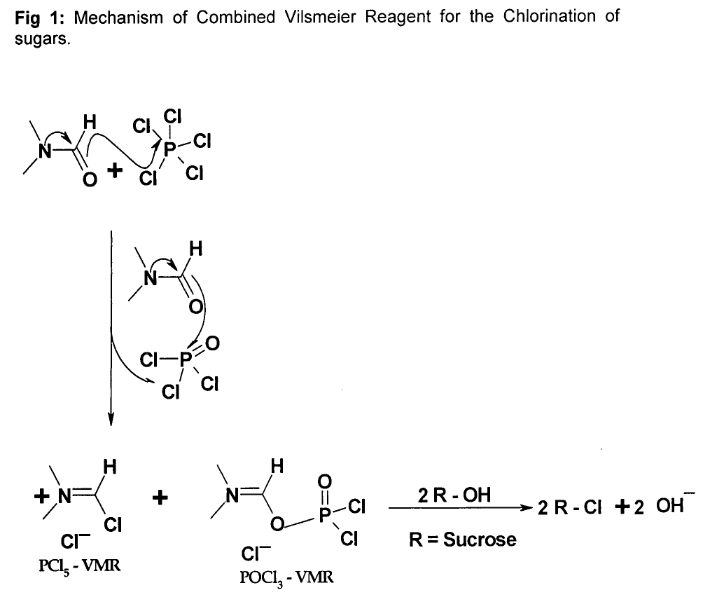

The possible mechanism of the reactions involved in the formation of combined Vilsmeier reagent from PCl₅ is elucidated in FIG. 1.

Total amount of 6-O-acylsucrose which could be thus chlorinated from same amount of PCl₅ was double than previously used methods in which by-product POCl₃ is removed from the reaction mixture after it is formed. This gives a new and more efficient way of using PCl₅ to chlorinate sucrose, its derivatives and for analogous chlorination reactions through the synthesis and application of Vilsmeier-Haack reagent without removal of the POCl₃ generated in-situ. This is a first instance where for chlorination reaction of sugar or its derivatives is driven by using a combined Vilsmeier-Haack reagent. Combined Vilsmeier-Haack Reagent, which may find use in chlorinating analogous and other organic molecules too, and all such reactions are embodiments of this invention.

The new method is a process where the solid Vilsmeier-Haack reagent is not isolated and is mixed with the Vilsmeier-Haack reagent formed with POCl₃ and taken up for chlorination. Thus where 10 moles of PCl₅ reacted with a tertiary amide such as DMF, 10 moles of Vilsmeier-Haack reagent along with 10 moles of POCl₃ are generated. The 10 moles of POCl₃ further react with available excess of DMF and form 10 moles of the second Vilsmeier-Haack reagent. Both the types of Vilsmeier-Haack reagent thus formed are contacted with 6.6 moles of substrate (sucrose-6-acetate) to carry out chlorination. The chlorination reaction was carried out by heating the reaction mixture to elevated temperatures and maintaining them at various temperatures for a required amount of time and then neutralizing at the end of the reaction by an appropriate base. The reaction efficiency evaluated as the quantity of TGS formed in such process was found to be almost double than that of the reaction with only PCl₅-Vilsmeier-Haack reaction. Effectively the substrate quantity was doubled for the same quantity of PCl₅ used for the reaction by not removing the POCl₃-Vilsmeier-Haack reagent formed as byproduct. This result has an economical implication towards the raw material cost and becomes highly profitable in the industrial process. Also the process of filtration of the solid Vilsmeier Haack reagent is avoided and reduces process costs.

### EXAMPLE 1

### Formation of Second Crop of Vilsmeier-Haack Reagent from Byproduct POCl₃ Formed from PCl₅ after Formation of First Crop of the Reagent

PCl₅, 835 g, was added to a round bottom flask containing 0.835 L of DMF at 20° C. The Vilsmeier-Haack reaction was accomplished indicated by the formation of white crystals of Vilsmeier-Haack reagent. After about 15 min, the liberated POCl₃ also started forming the Vilsmeier-Haack reagent and formed an orange red solution along with the solid. The mixture was then stirred thoroughly for 1.0 hr at room temperature. An excess of DMF, 500 ml, was added to the reaction. The mixture was cooled to 0° C. and the substrate containing 263 g of sucrose equivalent (sucrose-6-acetate) was added drop wise. The temperature was maintained below 0° C. during addition.

After the completion of addition of the substrate, the temperature was allowed to come to ambient and stirred for 1.0 hr. The temperature was then raised to 65° C., maintained for 1.5 hrs and further heated to 80° C. and maintained for 1.0 hr. Further the temperature was raised up to 115° C. and maintained for 3½ hrs. The reaction mass was then neutralized using calcium hydroxide slurry up to pH 7.0-7.5. The formation of TGS was evaluated by HPLC and was found to be 29% of the sucrose input

### EXAMPLE 2

### Chlorination by Vilsmeier-Haack Reagent Formed from PCl₅ Only

This experiment was carried out to show the efficiency of chlorination using only Vilsmeier-Haack reagent generated from PCl₅. 835 g of PCl₅ was added to a round bottom flask containing 0.835 L of DMF at 20° C. The Vilsmeier-Haack reaction was accomplished and was observed by the formation of white crystals of Vilsmeier-Haack reagent. The reaction was accompanied by the formation of POCl₃ which started to react with the available excess of DMF to form the second Vilsmeier-Haack reagent. But this Vilsmeier-Haack reagent that forms is in liquid form and doesn't become a solid Vilsmeier-Haack reagent as in the case of PCl₅. So, in order to ascertain and demonstrate efficacy of Vilsmeier-Haack reagent formed from PCl₅, the PCl₅ Vilsmeier-Haack reagent formed was filtered off and the POCl₃ and the excess DMF was separated out completely. The Vilsmeier-Haack reagent in solid form was washed with DMF and was taken up for the reaction.

The filtered Vilsmeier-Haack reagent crystals were taken in the reaction flask and care was taken to ensure there is no water contamination to the Vilsmeier-Haack reagent. 300 ml of DMF in excess was added to the Vilsmeier-Haack reagent and cooled to −5 to 0° C. The substrate containing 132 g of sucrose equivalent (sucrose-6-acetate) was added drop wise. The temperature was maintained below 0° C. during addition.

After the completion of addition of the substrate, the temperature was allowed to come to ambient and stirred for 1.0 hr. The temperature was then raised to 65° C., maintained for 1.5 hrs and further heated to 80° C. and maintained for 1.0 hr. Further the temperature was raised up to 115° C. and maintained for 3½ hrs. The reaction mass was then neutralized using calcium hydroxide slurry up to pH 7.0-7.5. The formation of TGS was evaluated by HPLC and was found to be 45% of sucrose input.

### EXAMPLE 3

### Chlorination by Vilsmeier-Haack Reagent Formed from POCl₃ Only

This experiment was carried out to show the efficiency of chlorination using only Vilsmeier-Haack reagent generated from POCl₃. 614.2 g of POCl₃ was added drop wise to a reaction flask containing 1250 ml of DMF. The temperature was maintained between 0 to 5° C. The formation of the Vilsmeier-Haack reagent was confirmed by the orange colour formation in the flask. The mixture was stirred for 1 hour for completion of the reagent formation and then the contents were cooled to 0 to −5° C. The substrate containing 132 g of sucrose equivalent (sucrose-6-acetate) was added drop wise. The temperature was maintained below 0° C. during addition.

After the completion of addition of the substrate, the temperature was allowed to come to ambient and stirred for 1.0 hr. The temperature was then raised to 65° C., maintained for 1.5 hrs and further heated to 80° C. and maintained for 1.0 hr. Further the temperature was raised up to 115° C. and maintained for 3½ hrs. The reaction mass was then neutralized using calcium hydroxide slurry up to pH 7.0-7.5. The formation of 4,1′, 6′trichlorogalactosucrose was evaluated by HPLC and was found to be 28% of sucrose input.

### EXAMPLE 4

### Removal of Byproduct POCl₃ from the First Vilmeier Reagent

835 g of PCl₅ was added to a round bottom flask containing 0.835 L of DMF at 80° C. under vacuum. The Vilsmeier-Haack reaction was accomplished and was observed by the formation of white crystals of Vilsmeier-Haack reagent. As the Vilsmeier reagent was being formed during the reaction, the POCl₃ evolved in the reaction was distilled off. The vapors of POCl₃ were condensed by a chiller and were recovered at the receiver end. The vacuum distillation was continued till the complete removal of POCl₃ from the reaction flask. DMF was continuously added in the reaction flask from time to time to facilitate complete removal of POCl₃ without the contents of the flask becoming dry.

Additional quantity of DMF was added in excess and then the reaction flask was cooled to −5-0° C. and 132 g of sucrose-6-acetate in DMF solution was added drop wise under constant stirring.

After the completion of addition of the substrate, the temperature was allowed to come to ambient and stirred for 1.0 hr. The temperature was then raised to 65° C., maintained for 1.5 hrs and further heated to 80° C. and maintained for 1.0 hr. Further the temperature was raised up to 115° C. and maintained for 3½ hrs. The reaction mass was then neutralized using calcium hydroxide slurry up to pH 7.0-7.5. The formation of 4,1′, 6′trichlorogalactosucrose was evaluated by HPLC and was found to be 20% of the sucrose input.

To the POCl₃ isolated by distillation and chilling, DMF was added and formation of Vilsmeier-Haack reagent was accomplished, indicated by formation of orange to red color. This reagent was, however, liquid, did not separate as crystals and was used in liquid condition only.

After converting the POCl₃ isolated by distillation and chilling to Vilsmeier reagent, 350 ml of additional quantity of DMF was added, the reaction flask was cooled to −5-0° C. and 400 g of sucrose-6-acetate in DMF solution was added drop wise under constant stirring.

After the completion of addition of the substrate, the temperature was allowed to come to ambient and stirred for 1.0 hr. The temperature was then raised to 65° C., maintained for 1.5 hrs and further heated to 80° C. and maintained for 1.0 hr. Further the temperature was raised up to 115° C. and maintained for 3½ hrs. The reaction mass was then neutralized using calcium hydroxide slurry up to pH 7.0-7.5. The formation of 4,1′, 6′trichlorogalactosucrose was evaluated by HPLC and was found to be - - - % of the sucrose input.

1. A process of preparation of Vilsmeier-Haack reagent from Phosphorus Pentachloride (PCl₅) comprising the steps of:
a. reacting N,N-dialkylformamide or N,N-dialkylacetamide, preferably N,N-dialkylformamide, more preferably N,N-dimethylformamide (DMF), with Phosphorus Pentachloride (PCl₅) to prepare a first crop of Vilsmeier reagent as insoluble crystals and Phosphorus Oxy-Chloride (POCl₃) as by-product,
b. allowing the said by-product POCl₃ to further react with DMF to form a second crop of Vilsmeier reagent in the same reaction mixture resulting into a combined Vilsmeier reagent, or,
c. isolating the said by-product POCl₃ from the first reaction mixture by one or more of a process of separation comprising distillation and cooling and this isolated POCl₃ is reacted with DMF to prepare second crop of Vilsmeier reagent; which is used for chlorination reaction
i. either independently and separately, or
ii. after combining with the said first crop of Vilsmeier reagent, or
iii. after combining with Vilsmeier reagent formed from reacting DMF with other sources of chlorinating agent.

2. A process of chlorinating a substrate, particularly a sucrose acylate by reacting the same under stirring and temperature control with a Vilsmeier reagent prepared by process of claim 1 and then heating and holding the reaction mixture to various temperatures for various periods of time until occurrence of desired degree of chlorination.

3. A process of claim 2 wherein:
a. the said sucrose acylate is sucrose-6-acetate or sucrose-6-benzoate, and
b. the reactants are added, stepwise,
i. preferably initially cooled, further preferably to below 0° C. to about −5° C.,
ii. mixed with each other taking care to keep it cool, preferably by drop wise addition to each other,
iii. allowing temperature to rise to ambient after completion of mixing of the reagents and stirring it further for about one hour,
iv. raising the temperature to about 65° C. and holding at that temperature for a period of time, preferably for about 1.5 hour,
v. raising the temperature to about 85° C. and holding at that temperature for a period of time, preferably for about one hour,
vi. raising the temperature to about 115° C. and holding at that temperature for a period of time, preferably for about 3.5 hours
vii. neutralizing the reaction mixture to about pH 7 to 7.5 by using alkali, preferably a calcium hydroxide slurry.
